(19) United States
(12) Patent Application Publication
(10) Pub. No.: US 2014/0366809 A1
(43) Pub. Date: Dec. 18, 2014

(54) ODOR CONTROL SYSTEMS WITH CUSTOMIZABLE AROMA

(71) Applicant: NESTEC SA, Vevey (CH)

(72) Inventors: Nathan Foster Huck, Jackson, MO (US); Mark Alan Donavon, Troy, IL (US)

(21) Appl. No.: 14/355,042

(22) PCT Filed: Oct. 5, 2012
(86) PCT No.: PCT/US12/58962

§ 371 Date: Apr. 29, 2014

Related U.S. Application Data
(60) Provisional application No. 61/628,650, filed on Nov. 3, 2011.

(51) Int. Cl. A61L 2/24; A01K 1/01; A61L 2/22

(52) U.S. Cl. CPC A61L 2/24; A61L 2/22; A01K 1/0107
USPC 119/163; 119/165; 119/169; 239/34

(57) ABSTRACT

The invention provides odor control systems having customizable aroma and malodor control compounds. In a general aspect, the odor control system includes a litter box and a programmable dissemination device attachable to the litter box. The programmable dissemination device is adaptable to receive at least one odor control cartridge and disseminate the contents of the cartridge. The odor control cartridge is a fragrance cartridge or a malodor counteractant cartridge insertable into the programmable dissemination device. Alternatively, the odor control cartridge can include a fragrance and a malodor counteractant in the same cartridge.

### CROSS REFERENCE TO RELATED APPLICATIONS

This application claims priority to U.S. Provisional Application Ser. No. 61/628,650 filed Nov. 3, 2011, the disclosure of which is incorporated herein by this reference.

### BACKGROUND OF THE INVENTION

1. Field of the Invention

The invention relates generally to odor control systems and particularly to odor control systems having customizable aroma and malodor control compounds.

2. Description of Related Art

Management of animal waste is a consequence of maintaining domestic animals in the home. To address this issue, litter boxes containing suitable particulate litters are employed. A critical attribute for an animal waste management solution is its ability to mitigate malodor, thus providing a more positive aroma experience for the user when attending to or cleaning the litter box. Consumers prefer litters that are perceived to reduce impressions of malodor as well as provide an appealing scent.

Animal waste management products such as animal litter are commonly used to collect and process animal waste, e.g., the waste of a pet such as a cat. The litter is poured into a litter box or tray and collects the animal's waste products. However, conventional litter boxes and animal litter contained in the litter box can be problematic for the user. For example, the animal litter may not typically absorb any substantial portion of the obnoxious odors produced by animal excrement. These odors caused by excessive waste can escape into the surrounding area and become an annoyance to the user and others.

Traditional litters attempt to enhance soiled aroma appeal by incorporating fragrances that mask the malodor. However, some consumers may find the scent of the fragrance itself objectionable. In addition, some consumers may prefer a stronger or a weaker scent intensity. Moreover, some consumers may prefer the intensity of the scent or the scent itself to change over time, or in response to some other input (e.g., cat elimination, motion, temperature, time of day, etc).

There is, therefore, a need for an odor control system that allows a user to customize their aroma experience and a method to employ such a system.

### SUMMARY OF THE INVENTION

It is, therefore, an object of the invention to provide odor control systems including a litter box and a programmable dissemination device attachable to the litter box.

It is another object of the invention to provide programmable dissemination devices for controlling odors.

It is still another object of the invention to provide customizable odor control cartridges for controlling odors.

It is yet another object of the invention to provide methods for managing odors from a litter box.

It is another object of the invention to provide kits having devices and instructions for controlling odors.

It is another object of the invention to provide packages and indicia describing the contents of the packages including an odor control system.

These and other objects are achieved using an odor control system including a litter box and a programmable dissemination device attachable to the litter box. The programmable dissemination device is adaptable to receive at least one odor control cartridge and disseminate the contents of the cartridge. The odor control cartridge can be a fragrance cartridge insertable into the programmable dissemination device. The odor control cartridge can also be a malodor counteractant cartridge insertable into the programmable dissemination device. The odor control system allows a user to customize the type, intensity, and timing of fragrance and/or malodor counteractant release from the cartridge.

Additional and further objects, features, and advantages of the invention will be readily apparent to those skilled in the art.

### BRIEF DESCRIPTION OF THE DRAWINGS

FIG. 1 shows an odor control system having a slot for a single odor cartridge in an embodiment of the invention.

FIG. 2 shows an odor control system having a dual slot for multiple odor control cartridges in an embodiment of the invention.

FIG. 3A shows a customizable odor control cartridge configured to be receivable by a programmable dissemination device in an embodiment of the invention.

FIG. 3B shows a cross-section IIIB-IIIB of the customizable odor control cartridge of FIG. 3A.

### DETAILED DESCRIPTION OF THE INVENTION

Definitions

The term “animal” means any animal that exhibits an odor. The animal can include a human, avian, bovine, canine, equine, feline, hicrine, lupine, murine, ovine, or porcine animal. The animal can also be any suitable pet or companion animal.

The term “companion animal” means domesticated animals such as cats, dogs, rabbits, guinea pigs, ferrets, hamsters, mice, gerbils, horses, cows, goats, sheep, donkeys, pigs, and the like, preferably dogs and cats, most preferably cats.

The term “litter(s)” means a composition that is suitable for use as an animal litter (e.g., managing animal waste) but that can also be used for any other suitable purpose. For example, a litter described herein could be used to absorb a chemical spill, absorb an oil spill, create traction on a slippery surface, and the like.

The term “single package” means that the components of a kit are physically associated in or with one or more containers and considered a unit for manufacture, distribution, sale, or use. Containers include, but are not limited to, bags, boxes, cartons, bottles, packages of any type or design or material, over-wrap, shrink-wrap, affixed components (e.g., stapled, adhered, or the like), or combinations thereof. A single package may contain one or more of a litter box, programmable dissemination device, odor control cartridge, or the like as described herein that can be physically associated such that they are considered a unit for manufacture, distribution, sale, or use.

The term “virtual package” means that the components of a kit are associated by directions on one or more physical or virtual kit components instructing the user how to obtain the other components, e.g., a bag or other container containing one component and directions instructing the user to go to a website, contact a recorded message or a fax-back service, view a visual message, or contact an instructor to obtain instructions on how to use the kit or safety or technical information about one or more components of a kit.

All percentages expressed herein relating to the components of a composition are by weight of the total weight of the composition unless expressed otherwise.

As used throughout, ranges are used herein in shorthand, to avoid having to set out at length and describe each and every value within the range. Any appropriate value within the range can be selected, where appropriate, as the upper value, lower value, or the terminus of the range.

As used herein and in the appended claims, the singular form of a word includes the plural, and vice versa, unless the context clearly dictates otherwise. Thus, the references “a”, “an”, and “the” are generally inclusive of the plurals of the respective terms. For example, reference to “an odor control system” or “a method” includes a plurality of such “odor control systems” or “methods”. Similarly, the words “comprise”, “comprises”, and “comprising” are to be interpreted inclusively rather than exclusively. Likewise, the terms “include”, “including” and “or” should all be construed to be inclusive, unless such a construction is clearly prohibited from the context. Where used herein the term “examples,” particularly when followed by a listing of terms is merely exemplary and illustrative, and should not be deemed to be exclusive or comprehensive.

The devices, assemblies, kits, methods, compositions and other advances disclosed here are not limited to particular methodology, protocols and reagents described herein because, as the skilled artisan will appreciate, they may vary. Further, the terminology used herein is for the purpose of describing particular embodiments only, and is not intended to, and does not, limit the scope of that which is disclosed or claimed.

The Invention

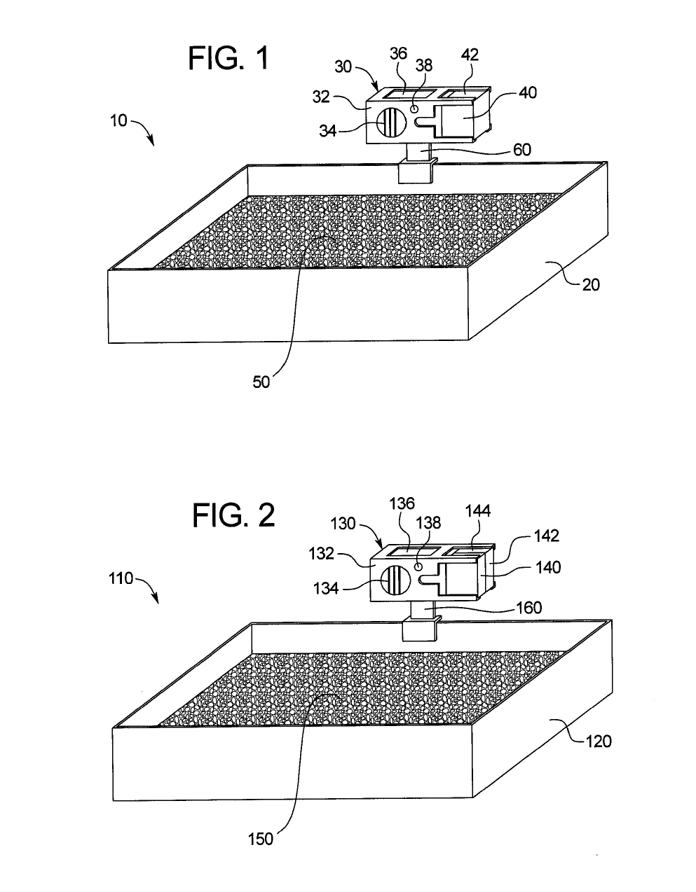

In one aspect, illustrated in FIG. 1, the invention provides an odor control system 10 including a litter box 20 and a programmable dissemination device 30 attachable to litter box 20. Litter box 20 can be any suitable shape and be sized to hold a sufficient amount of animal litter for a specified use as well as be designed for easy access by an animal, e.g., based on the size of the animal or frequency of use by one or more animals.

Programmable dissemination device 30 includes a housing 32 adaptable to receive an odor control cartridge 40 partially or fully within a slot 42 of housing 32. Programmable dissemination device 30 can be attached to litter box 20 using any suitable attachment mechanism 60 (e.g., clip-on, VELCRO® Brand Fasteners or their equivalent, snap-fit, adhesive, etc.). Programmable dissemination device 30 and/or litter box 20 can be specifically designed so that programmable dissemination device 30 has a custom fit with litter box 20. Alternatively, programmable dissemination device 30 can be integrally formed with litter box 20 as a single apparatus.

Programmable dissemination device 30 is has the necessary components to be configured to disseminate or release the contents of an odor control cartridge 40. Programmable dissemination device 20 can include an outlet 34 for disseminating the contents of odor control cartridge 40. Outlet 34 can be directed towards litter box 20 or away from litter box 20 (e.g., facing upward). In an embodiment, outlet 34 can be adjustable to face any suitable direction desired by a user. This allows the user to disseminate the contents of odor control cartridge 40 in any direction as desired.

Programmable dissemination device 30 can further include a programmable interface 36 for programming device 30. Programmable dissemination device 30 can be programmed to release a compound from odor control cartridge 40 based on any suitable factors such as a specific time, frequency, temperature, duration or a combination thereof. For example, specific programmable elements could include release based on time of day, duration of release, and/or cartridge selection criteria (e.g., disseminate contents from one or more cartridges based on when the animal uses the litter box—or release from a malodor counteractant cartridge only after cat elimination).

Programmable dissemination device 30 can be programmable via a remote transmitter. For example, programmable dissemination device 30 can include a sensor 38 that operates programmable dissemination device 30 when receiving a signal from a remote transmitter, e.g., a remote control device, a telephone ringer, a transmitter connected to the internet, a transmitter activated by telephone (e.g., cell phone), a wi-fi device (e.g., a smart phone, tablet computer), animal collar, and the like. In another embodiment, programmable dissemination device 30 can include its own transmitter to communicate with external devices, e.g., send information to a receiver indicating that the litter amount in litter box 20 is lower than desirable.

Sensor 38 can also be configured to detect the presence of an animal in or proximate to litter box 20. In this manner, programmable dissemination device 30 can be programmed to disseminate the contents of odor control cartridge 40 when sensor detects an animal approaching or inside litter box 20. Alternatively or in addition to, programmable dissemination device 30 can be programmed to disseminate the contents of odor control cartridge 40 at a specific time, frequency, and/or duration before, during and/or after sensor detects an animal approaching or inside litter box 20. In a further embodiment, sensor 38 operates programmable dissemination device 30 after a use of litter box 20 by an animal, e.g., sensor 38 senses the presence of the animal or the presence of a transmitter implanted in the animal or an article associated with the animal, e.g., a collar, harness, or piece of clothing worn by the animal.

In an alternative embodiment, programmable dissemination device 30 can be a stand-alone device that is adapted to be positioned at or near a litter box or at any location desired by a user. In this manner, programmable dissemination device 30 can include any suitable attachment mechanism (e.g., clip-on, hook, VELCRO® Brand Fasteners or their equivalent, etc.) to allow a user flexibility in where to locate programmable dissemination device 30 with respect to an odor source.

Odor control cartridge 40 is configured to be receivable by programmable dissemination device 30 in any manner known to the skilled artisan. Although shown having a box shape in FIG. 1, odor control cartridge 40 can have any suitable shape (e.g., cylinder, cube, cuboid, etc.) and size to be receivable by programmable dissemination device 30.

Odor control cartridge 40 can also have any suitable mechanism (e.g., outlet, puncturable membrane, piston, etc.) known to the skilled artisan to assist in accessing the odor control compound contained therein to be disseminated by programmable dissemination device 30. In alternative embodiments, the odor control compound can be contained partially (e.g., to have a portion exposed to the atmosphere) or completely within odor control cartridge 40. Programmable dissemination device 30 can have any suitable dissemination mechanism known to the skilled artisan by which to disseminate the contents of odor control cartridge 40. Suitable dissemination mechanisms include a plunger, squeezing, spraying, heating, etc.

The odor control compound can be released directly from odor control cartridge 40 into the air or toward litter box 20 without passing through programmable dissemination device 30. Rather than receiving the odor control compound, programmable dissemination device 30 can incorporate a dissemination mechanism that causes the odor control compound to be disseminated directly from odor control cartridge 40. For example, odor control cartridge 40 can have a partially exposed odor control compound that is released when heated by programmable dissemination device 30.

In an embodiment, odor control cartridge 40 is a fragrance cartridge insertable into slot 42 of programmable dissemination device 30. The fragrances used in odor control cartridge 40 for programmable dissemination device 30 can be any fragrances useful for combating litter box odors. Many such fragrances are known to the skilled artisan. Such fragrances include, but are not limited to, compounds such as alicate, pyralone, limonene, linalool, β-phenethyl alcohol, and β-myrcene, nonadienal, ambrofix, amyl vinyl carbinol, and compositions or “cocktails” such as botanical oils and essences. Others include fragrances selected from esters, linear terpenes, cyclic terpenes, aromatics, amines, alcohols, aldehydes, esters, ketones, lactones, and thiols.

In another embodiment, odor control cartridge 40 is a malodor counteractant cartridge insertable into slot 42 of programmable dissemination device 30. The malodor counteractants (“MOCs”) used in odor control cartridge 40 for programmable dissemination device 30 can be any MOCs useful for combating litter box odors. Many such MOCs are known to the skilled artisan. Such MOCs include, but are not limited to, chlorinated oxidizers (e.g., chlorine dioxide), aldehydes, permanganates, peroxides, metal oxides, borates, borax, perborates, enzymes, urease inhibitors, proteases, cationic surfactants (e.g., N-ethyl-N-soyamorpholiniumethosulphate), metallic salts (e.g., zinc salts, zinc ricinoleate, zinc chloride, zinc gluconate), metal ions (e.g., transition metal ions), nano-particulate metal ions (e.g., ferric and aluminum salts), silver, copper, zinc, carbon (e.g., activated), oxides, cyclodextrins, zeoloites, activated carbon, activated alumina, calcium carbonate, silicas, clay minerals, chlorophyll, metal organic frameworks (“MOFs”), molecular sieves, and chelating agents and metal binders such as EDTA or any combinations thereof.

In an alternative embodiment, odor control cartridge 40 can include more than one fragrance and/or MOC within the same cartridge. Programmable dissemination device 30 can then be programmed for selecting and releasing specified amounts and/or blends of fragrances and MOCs from odor control cartridge 40. Accordingly, the intensity of the fragrances and MOCs in odor control cartridge 40 can be controlled (e.g., through increasing or decreasing amount of fragrances and MOCs released from the cartridge).

As further illustrated in FIG. 1, litter box 20 of odor control system 10 can contain a litter 50 to directly absorb waste products of an animal that is using litter box 20. Litter 50 can be in the form of clumping litter, non-clumping litter, inorganic litter, organic litter, paper, sawdust, or a combination thereof. Litter 50 can have a fragrance or be unscented. In an alternative embodiment, litter box 20 can include an absorbent pad in addition to or in place of a litter.

In an alternative aspect, illustrated in FIG. 2, the invention provides an odor control system 110 including a litter box 120 and a programmable dissemination device 130 attachable to litter box 120. Programmable dissemination device 130 includes a housing 132 adaptable to receive a plurality of odor control cartridges 140 and 142 partially or fully within one or more slots 144 of housing 132. Although two odor control cartridges are shown in FIG. 2, programmable dissemination device 130 can be configured to receive more than two odor control cartridges.

Programmable dissemination device 130 has the necessary components to be configured to disseminate or release the contents of odor control cartridges 140 and 142 in any suitable order or amount. Programmable dissemination device 120 can include an outlet 134 for disseminating the contents of odor control cartridges 140 and 142. Outlet 134 can be directed towards litter box 120 or away from litter box 120 (e.g., facing upward). In an embodiment, outlet 134 can be adjustable to face any suitable direction desired by a user.

Programmable dissemination device 130 can further include a programmable interface 36 for programming device 130. Programmable dissemination device 130 can be programmed to release a compound from odor control cartridge 140 based on any suitable factors such as a specific time, frequency, temperature, duration or a combination thereof. Programmable dissemination device 130 can also be programmed for selecting and releasing specified amounts and/or blends of fragrances and MOCs from one or more of odor control cartridges 140 and 142. Accordingly, the intensity of the fragrances and MOCs odor control cartridges 140 and 142 can be controlled (e.g., through increasing or decreasing amount of fragrances and MOCs released from the cartridge).

Programmable dissemination device 130 can include a sensor 138 that operates programmable dissemination device 130 when receiving a signal from a remote transmitter as discussed in previous embodiments. Sensor 138 can also be configured to detect the presence of an animal in or proximate to litter box 120 as discussed in previous embodiments.

Programmable dissemination device 130 can be attached to litter box 120 using any suitable attachment mechanism 160 (e.g., clip-on, VELCRO® Brand Fasteners or their equivalent, snap-fit, adhesive, etc.). Litter box 120 of odor control system 110 can contain any suitable litter 150 and/or absorbent pad to directly absorb waste products of an animal that is using litter box 120.

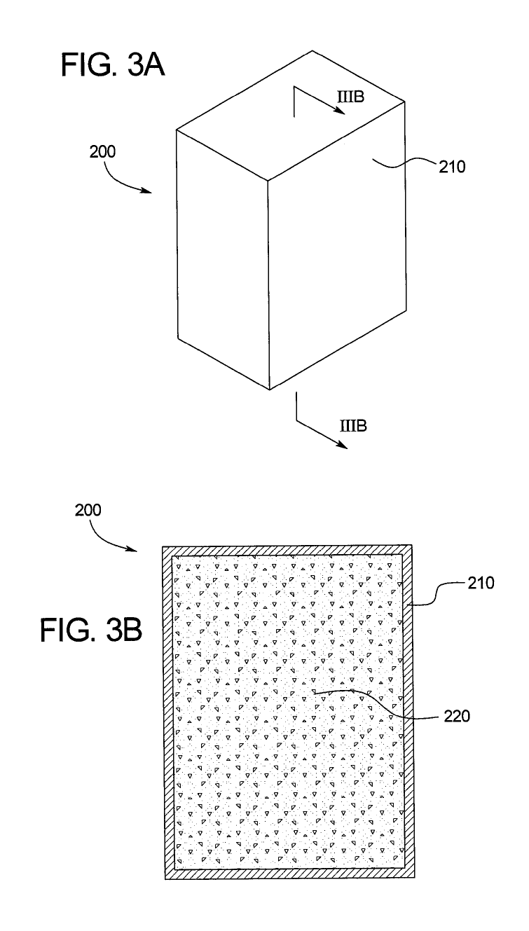

In another aspect, illustrated in FIGS. 3A-3B, the invention provides a customizable odor control cartridge 200 including a housing 210 and at least one odor control compound 220 contained in housing 210. Customizable odor control cartridge 200 is configured to be receivable by a programmable dissemination device according to any of the embodiments described herein.

Although shown having a box shape in FIGS. 3A-3B, customizable odor control cartridge 200 can have any suitable shape and size to be receivable by the programmable dissemination device. Customizable odor control cartridge 200 can also have any suitable mechanism (e.g., outlet, puncturable membrane, plunger, piston, etc.) known to the skilled artisan by which odor control compound 220 is released from customizable odor control cartridge 200. In an embodiment, odor control compound 220 can be contained partially (e.g., to have a portion exposed to the atmosphere) or completely within housing 210. In another embodiment, customizable odor control cartridge 200 can include one or more fragrances and/or MOCs within a single cartridge.

Customizable odor control cartridge 200 can be sold individually in a package or in multi-packs. In this manner, a user can individually purchase one or more customizable odor control cartridges 200 to be used with the programmable dissemination device. If sold in multi-packs, different customizable odor control cartridges can be sold together based on a specific combination of fragrances or MOCs contained in each cartridge. The package can include indicia describing the customizable odor control cartridge configured to be receivable by a programmable dissemination device according to any of the embodiments described herein. The indicia can be in the form of words, symbols, pictures, photographs, figures, or combinations thereof to show details or examples of the odor control cartridges described therein. The package can further contain or more of the customizable odor control cartridges configured to be receivable by a programmable dissemination device according to any of the embodiments described herein.

The customizable odor control cartridges could be purchased from a store, online, or from a print catalog directly from the manufacturer. Sample cards with scratch and sniff elements related to particular customizable odor control cartridges could be available at the store or distributed through direct mailings to assist consumers in selecting desired customizable odor control cartridges.

In another aspect, the present invention provides a method of managing odor from a litter box. The method comprises providing a programmable dissemination device located on or proximately to a litter box. The programmable dissemination device is adaptable to receive at least one odor control cartridge and disseminate the contents of the cartridge. The method further comprises inserting at least one odor control cartridge into the programmable dissemination device and releasing an odor control compound from the cartridge.

According to an embodiment of the method, the programmable dissemination device can be programmed to release an odor control compound from the cartridge based on a factor such as time, frequency, temperature, duration or a combination thereof. Alternatively, or in addition to, the programmable dissemination device can be programmed to release an odor control compound from the cartridge based on detecting the presence of an animal in the litter box. The dissemination device can also be programmed to release an odor control compound from the cartridge based on a remote transmitter such as a remote control device, cell phone, internet, animal collar, wi-fi device, or combinations thereof.

In the various embodiments, the programmable dissemination device, sensor, programmable interface, and similar components can be powered by any suitable means, e.g., photoelectric cell, solar panel, battery, electricity, or other suitable means. In preferred embodiments, the programmable dissemination device, sensor, programmable interface, and similar components are powered by electricity or batteries, including rechargeable batteries.

In an alternative aspect, the invention provides kits useful for controlling odor and using the odor control systems of the invention. The kits include in a single package or in separate containers in a virtual package, as appropriate for the kit component, either (A) a litter box and a programmable dissemination device attachable to the litter box according to any of the embodiments described herein; or (B) a programmable dissemination device adaptable to receive at least one odor control cartridge and disseminate the contents of the cartridge, and at least one of (1) a litter box; (2) litter; (3) an absorbent pad; (4) a fragrance cartridge; (5) a malodor counteractant cartridge; (6) an odor control cartridge having a fragrance and a malodor counteractant; (7) a remote transmitter; (8) an attachment device for attaching the programmable dissemination device to an object; (9) instructions on how to program the programmable dissemination device; (10) instructions on how to control odor using the programmable dissemination device; (11) instructions on how to properly dispose of the litter, or (12) one or more batteries.

When the kits comprise a virtual package, the kits are limited to instructions in a virtual environment in combination with one or more physical kit components. The kits may contain the kit components in any of various combinations. In one embodiment, the kit contains a litter box and a programmable dissemination device attachable to the litter box. In addition, one or more odor control cartridges can be sold with this kit or sold separately from the kit, for example, as part of a virtual kit.

The kits can encompass one or more kit components that are ordered and shipped separately to a consumer, for example, such as an order on the internet or by phone for a litter box and a programmable dissemination device, wherein the two articles are shipped from separate locations to the consumer's address. In an embodiment, the programmable dissemination device can be constructed and arranged to attach to any suitable object to control odors in the proximate area of the programmable dissemination device.

In a further aspect, the invention provides a means for communicating information about or instructions for one or more of (1) assembling a litter box and a programmable dissemination device to produce an odor control system; (2) using a fragrance cartridge with the odor control system to control odor in the litter box; (3) using a malodor counteractant cartridge with the odor control system to control odor in the litter box; (4) using an odor control cartridge having a fragrance and a malodor counteractant with the odor control system to control odor in the litter box; (5) programming the programmable dissemination device to control odor in the litter box; (6) remotely programming the programmable dissemination device to control odor in the litter box; or (7) a list of programs to use with the odor control system.

The communication means can be a document, digital storage media, optical storage media, audio presentation, or visual display containing the information or instructions. In certain embodiments, the communication means can be a displayed website, a visual display kiosk, a brochure, a product label, a package insert, an advertisement, a handout, a public announcement, an audiotape, a videotape, digital streaming, a DVD, a CD-ROM, a computer readable chip, a computer readable card, a computer readable disk, a USB device, a FireWire device, a computer memory, and any combination thereof.

Useful information includes one or more of contact information for consumers to use if they have a question about the invention and its use. The communication means is useful for instructing on the benefits of using the present invention and communicating the approved methods for using the odor control system for controlling odors, for example, from a litter box.

In another aspect, the invention provides a package including indicia describing an odor control system including a litter box and a programmable dissemination device attachable to the litter box according to any of the embodiments described herein. The indicia can be in the form of words, symbols, pictures, photographs, figures, or combinations thereof to show details or examples of the odor control system described therein. The package can further contain an odor control system including a litter box and a programmable dissemination device attachable to the litter box according to any of the embodiments described herein.

The package can include one or more handles suitable for handling and transporting the package. The package can include one or more windows for viewing the odor control system or any parts thereof. The package can include a label affixed to the package containing a word or words, picture, design, acronym, slogan, phrase, or combination thereof, that indicate that the package contains an odor control system including a litter box and a programmable dissemination device attachable to the litter box according to any of the embodiment described herein.

All patents, patent applications, publications, and other references cited or referred to herein are incorporated herein by reference to the extent allowed by law. The discussion of those references is intended merely to summarize the assertions made therein. No admission is made that any such patents, patent applications, publications or references, or any portion thereof, are relevant prior art for the present invention and the right to challenge the accuracy and pertinence of such patents, patent applications, publications, and other references is specifically reserved.

What is claimed is:

1. An odor control system comprising:
a litter box; and
a programmable dissemination device attachable to the litter box, the programmable dissemination device adaptable to receive at least one odor control cartridge and disseminate a content of the cartridge.

2. The odor control system of claim 1 wherein the programmable dissemination device includes a sensor for detecting the presence of an animal in the litter box.

3. The odor control system of claim 1 wherein the odor control cartridge comprises a fragrance cartridge insertable into the programmable dissemination device.

4. The odor control system of claim 3 wherein the fragrance cartridge stores at least one fragrance selected from the group consisting of alicate, pyralone, limonene, linalool, β-phenethyl alcohol, and β-myrcene, nonadienal, ambrofix, amyl vinyl carbinol, botanical oils, botanical essences, and combinations thereof.

5. The odor control system of claim 1 wherein the odor control cartridge comprises a malodor counteractant cartridge insertable into the programmable dissemination device.

6. The odor control system of claim 5 wherein the malodor counteractant cartridge stores at least one malodor counteractant selected from the group consisting of chlorinated oxidizers, aldehydes, permanganates, peroxides, metal oxides, borates, borax, perborates, enzymes, urease inhibitors, proteases, cationic surfactants, metallic salts, zinc salts, zinc ricinoleate, zinc chloride, zinc gluconate, metal ions, transition metal ions, nano-particulate metal ions, ferric salts, aluminum salts, silver, copper, zinc, carbon, oxides, cyclodextrins, zeoloites, activated carbon, activated alumina, calcium carbonate, silicas, clay minerals, chlorophyll, metal organic frameworks, molecular sieves, chelating agents, metal binders, and combinations thereof.

7. The odor control system of claim 1 wherein the odor control cartridge comprises at least one fragrance and at least one malodor counteractant.

8. (canceled)

9. (canceled)

10. A programmable dissemination device comprising a housing adaptable to receive at least one odor control cartridge and disseminate a content of the cartridge.

11. The programmable dissemination device of claim 10 wherein the programmable dissemination device is programmed to release a compound from the cartridge based on a factor selected from the group consisting of time, frequency, temperature, duration, and combinations thereof.

12. The programmable dissemination device of claim 10 wherein the programmable dissemination device includes a sensor for detecting the presence of an animal.

13. The programmable dissemination device of claim 10 wherein the programmable dissemination device is programmable via a remote transmitter.

14. The programmable dissemination device of claim 13 wherein the remote transmitter is selected from the group consisting of remote control device, cell phone, internet, animal collar, wi-fi device, and combinations thereof.

15. The programmable dissemination device of claim 10 wherein the housing is attachable to a litter box.

16. A programmable dissemination device comprising a housing and including at least one odor control cartridge receivable by the housing, the programmable dissemination device adapted to disseminate a content of the cartridge.

17. The programmable dissemination device of claim 16 wherein the odor control cartridge comprises a fragrance cartridge insertable into the programmable dissemination device.

18. The programmable dissemination device of claim 16 wherein the odor control cartridge comprises a malodor counteractant cartridge insertable into the programmable dissemination device.

19. The programmable dissemination device of claim 16 wherein the programmable dissemination device is programmed to release a compound from the cartridge based on a factor selected from the group consisting of time, frequency, temperature, duration, and combinations thereof.

20. The programmable dissemination device of claim 16 wherein the programmable dissemination device is programmable via a remote transmitter.

21. The programmable dissemination device of claim 20 wherein the remote transmitter is selected from the group consisting of remote control device, cell phone, internet, animal collar, wi-fi device, and combinations thereof.

22. The programmable dissemination device of claim 16 wherein the housing is attachable to a litter box.

23. A customizable odor control cartridge comprising a housing and at least one odor control compound contained within the housing, the customizable odor control cartridge configured to be receivable by a programmable dissemination device.

24. The customizable odor control cartridge of claim 23 wherein the odor control compound comprises a fragrance.

25. The customizable odor control cartridge of claim 23 wherein the odor control compound comprises a malodor counteractant.

26. The customizable odor control cartridge of claim 23 wherein the odor control compound comprises at least one fragrance and at least one malodor counteractant.

27. An odor control system comprising:
a litter box;
at least one of a litter or an absorbent pad contained in the litter box;
at least one odor control cartridge; and
a programmable dissemination device attachable to the litter box, the programmable dissemination device adaptable to receive the at least one odor control cartridge and disseminate a content of the cartridge.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)
